(19) United States
(12) Patent Application Publication
(10) Pub. No.: US 2025/0339170 A1
(43) Pub. Date: Nov. 6, 2025

(54) MANAGEMENT OF STORED ANGULAR MOMENTUM IN STALLED INTRAVASCULAR ROTATIONAL DRIVE SHAFTS FOR ATHERECTOMY

(71) Applicant: CARDIOVASCULAR SYSTEMS, INC., St. Paul, MN (US)

(72) Inventors: Joseph P. Higgins, Wayzata, MN (US); Matthew W. Tilstra, Rogers, MN (US); Jeffrey R. Stone, Minnetonka, MN (US)

(21) Appl. No.: 18/866,415

(22) PCT Filed: May 19, 2022
(86) PCT No.: PCT/US2022/072437

§ 371 Date: Nov. 15, 2024

(51) Int. Cl. A61B 17/3207; A61B 17/00; A61B 90/00

(52) U.S. Cl. CPC A61B 17/320758; A61B 90/03; A61B 2017/00039; A61B 2017/00398; A61B 2090/031; A61B 2090/066

(57) ABSTRACT

A control system for releasing stored rotational energy and angular momentum in a drive shaft with a distally positioned atherectomy tool, wherein the atherectomy tool is stuck within vasculature. The control system is configured to manage the release of the stored energy and angular momentum of the drive shaft safely and predictably.

### CROSS-REFERENCE TO RELATED APPLICATIONS

None

### STATEMENT REGARDING FEDERALLY SPONSORED RESEARCH OR DEVELOPMENT

Not Applicable

### BACKGROUND OF THE INVENTION

### Field of the Invention

Rotational atherectomy medical devices configured for intravascular use and having an external motor configured to rotatingly drive a rotational drive shaft having an atherectomy tool at or near a distal end of the drive shaft. More specifically, management of angular momentum stored in a stalled rotational drive shaft.

### Description of the Related Art

Rotational atherectomy is a non-surgical procedure to open blocked coronary arteries or vein grafts by using a device on the end of a catheter to cut or shave away atherosclerotic plaque (a deposit of fat and other substances, including hardened or calcified material, that accumulates on or within the lining of the artery wall).

Rotational atherectomy may be performed to restore the flow of oxygen-rich blood to the heart, to relieve chest pain, and to prevent heart attacks. It may be done on patients with chest pain who have not responded to other medical therapy and on certain of those who are candidates for balloon angioplasty (a surgical procedure in which a balloon catheter is used to flatten plaque against an artery wall) or coronary artery bypass graft surgery as well as peripheral artery treatments. It is sometimes performed to remove plaque that has built up after a coronary artery bypass graft surgery.

Rotational atherectomy may use a tool placed on the end of a rotational drive shaft to remove the plaque. Rotational atherectomy uses the rotating tool to grind up plaque and may be used instead of, or in conjunction with, balloon angioplasty or lithoplasty.

Several devices have been disclosed that perform rotational atherectomy. For instance, U.S. Pat. No. 5,360,432, issued on Nov. 1, 1994 to Leonid Shturman, and titled “Abrasive drive shaft device for directional rotational atherectomy” discloses an abrasive drive shaft atherectomy device for removing stenotic tissue from an artery, and is incorporated by reference herein in its entirety. The device includes a rotational atherectomy apparatus having a flexible, elongated drive shaft having a central lumen and a segment, near its distal end, coated with an abrasive material to define an abrasive segment. At sufficiently high rotational speeds, the abrasive segment expands radially, and can sweep out an abrading diameter that is larger than its rest diameter. In this manner, the atherectomy device may remove a blockage that is larger than the catheter itself. Use of an expandable head is an improvement over atherectomy devices that use non-expandable heads; such non-expandable devices typically require removal of particular blockages in stages, with each stage using a differently-sized head.

U.S. Pat. No. 5,314,438 (Shturman) shows another atherectomy device having a rotatable drive shaft with a section of the drive shaft having an enlarged diameter, at least a segment of this enlarged diameter section being covered with an abrasive material to define an abrasive segment of the drive shaft. When rotated at high speeds, the abrasive segment is capable of removing stenotic tissue from an artery.

Generally, rotational medical devices for intravascular use comprise an externally located motor with a driven rotational drive shaft attached thereto and an atherectomy tool at or near a distal end of the drive shaft, e.g., an atherectomy device as, e.g., in U.S. Pat. No. 6,494,890 incorporated herein by reference in its entirety. An exemplary prior art rotational atherectomy device is illustrated in FIG. 1. The prior art atherectomy device of FIG. 1 includes a handle portion 10, an elongated, flexible and rotational drive shaft 20 having an atherectomy tool 28 disposed on or near a distal end of drive shaft 20, and an elongated catheter 13 extending distally from the handle portion 10. The drive shaft 20 may be constructed from helically coiled wire as is known in the art and the atherectomy tool 28 may be fixedly attached thereto. The catheter 13 has a lumen configured to allow translation of drive shaft 20 therethrough and rotation of the drive shaft 20 therein. The drive shaft 20 may also comprise an inner lumen, permitting the drive shaft 20 to be advanced and rotated over a guide wire 15. A fluid supply line 17 may be provided for introducing a cooling and lubricating solution (typically saline or another biocompatible fluid) into the catheter 13.

The handle 10 desirably contains an electric motor (not shown and is well known in the art) for rotating the drive shaft 20 at high speeds, wherein a proximal end of the drive shaft 20 is operatively connected with the electric motor. Thus, rotation of the electric motor's connection to the drive shaft 20 causes rotation of the drive shaft 20. The handle 10 typically may be connected to a power source, such as an electrical outlet to power the electric motor. Hall sensors may be used to monitor rotational speed and/or rotational position of the electric motor. The handle 10 may also include a control knob 11 for advancing and retracting the electric motor and drive shaft 20 with respect to the catheter 13 and the body of the handle.

During the atherectomy treatment, the rotating atherectomy tool may become stuck within the subject lesion, a condition known as a “stall” in the art. In some known systems, when a stall condition occurs, the driving motor continues to apply torque to the proximal end of the drive shaft for a period of time until the blockage or stall condition is detected. This results in a considerable build-up of angular momentum and related energy within the wound-up drive shaft. Management of this energy is critical to ensuring that the vasculature is not injured.

Some known systems, e.g., those with electric motors, may stop the motor upon detecting a stall, and then allow the drive shaft to release the angular moment and related stored energy by unwinding in essentially an uncontrolled manner, wherein the electric motor may be manually turned in a reverse direction as a result of the unwinding drive shaft. This is undesirable in several ways, including potential trauma during the initial uncontrolled unwinding. FIG. 2 thus illustrates an embodiment that detects a stall condition, stops the driving motor, but continues to apply a “forward” rotational torque to the proximal end of the drive shaft, but at a torque level that may be overcome by the unwinding drive shaft to slowly manually and partially reverse the rotation of the motor to help slowly release the stored energy in the stalled drive shaft. As can be seen, applied torque is not allowed to reduce to zero in this embodiment, nor is the drive shaft completely unwound, which may result in trauma in certain cases wherein the blockage suddenly releases the atherectomy tool disposed on the partially unwound drive shaft resulting in an uncontrolled and rapid unwinding. In some of these cases, a phenomenon known as “jumping” may occur wherein the unwinding causes the drive shaft to lengthen, rapidly translating the atherectomy tool in the distal direction and further into the vasculature in an uncontrolled manner.

Other prior art atherectomy systems may detect a stall condition with subsequent release of the motor, wherein the motor is allowed to freely spin. FIG. 3 shows such a control mechanism wherein the applied torque of the motor to the proximal end of the drive shaft is allowed to become zero and the angular momentum of the stalled drive shaft is dissipated as a result. See, e.g., US 2011/021339.

In some known systems, a phenomenon known in the art as “ringing” may occur wherein the drive shaft cycles through unwinding and winding in order to release the stored energy and angular momentum. The “ringing” is substantially uncontrolled which, for reasons described above, may cause trauma to the vasculature. In some cases, the “ringing” drive shaft may lengthen and shorten as it experiences energy release through uncontrolled unwindings and windings, which may also cause trauma to the vasculature.

In other known control systems, an impending stall condition may be predicted (before stall condition actually occurs), wherein the atherectomy tool at or near the distal end of the rotating drive shaft would have zero rotational speed. The prediction of a stall condition is effected whenever the load on the motor and/or drive shaft exceeds a threshold value which, in turn, causes an automatic reversal of the rotational direction of the drive shaft. See, e.g., U.S. Pat. No. 9,820,770. Such control systems are focused on avoiding stall conditions, not releasing angular momentum and/or stored energy resulting from actual stall conditions.

Similarly, U.S. Pat. No. 11,172,956 predicts a stall condition with subsequent adjustment of the speed of rotation, thus focusing on stall avoidance and minimizing future build-up of angular momentum and/or stored energy should the stall actually occur.

And, U.S. Pat. No. 11,291,468 discloses mechanisms for overcoming a stall condition, i.e., a stuck atherectomy burr. Such mechanisms include alternating rotational directions and/or at a torque greater than a predetermined torque limit. Each such mechanism is concerned with helping the burr become unstuck, but there is no disclosure of releasing any stored energy or angular momentum within the drive shaft during the stall condition.

There are two stall detection methods present in known commercially available atherectomy systems:
1. If, at any time during electric motor startup (ramping up to a target rotational speed) or normal motor operation, the motor does not rotate for a period of time T (specifically, if the atherectomy system does not detect any motor rotor position changes during this period—i.e. the rotational speed is zero), the atherectomy system considers this a stall condition and stops delivering power to the motor.
2. If, after the electric motor should have ramped up to target rotational speed, the motor's speed drops below P percent of the motor's target speed for more than D amount of time, the atherectomy system considers this a stall condition and stops delivering to the motor.

In the known systems, the drive shaft itself may be damaged by a stall condition as the motor continues to rotate at least several turns before the stall condition is detected and the motor is signaled or instructed to react as above. As noted, the stalled drive shaft will wind up and store rotational energy and/or angular momentum that will need to be released. Uncontrolled unwinding, e.g., may cause damage to the drive shaft wherein the distal portion of the drive shaft (at the atherectomy tool location) till be held in place by the lesion or whatever structure has caused the stall while the still-rotating proximal end winds up, the motor senses the stall and stops with unwinding of the drive shaft occurring first at the proximal or motor side of the drive shaft where drive shaft damage is most likely to occur. FIGS. 4A and 4B are photographs of exemplary drive shaft damage observed as a result of winding, unwinding and rewinding following a stall condition. The drive shaft wire filars have become unwound and/or raised above the nominal outer diameter of the undamaged drive shaft, and further appear to have been rewound in the opposite direction resulting in a non-uniform and distorted profile.

Thus, it would be advantageous to provide a control mechanism that, among other things, provides a controlled and predictable unwinding of the stalled and wound drive shaft to release the stored rotational energy and angular momentum therefrom in order to prevent damage to the drive shaft and the patient's vasculature.

The various inventions disclosed herein address these, inter alia, issues.

### BRIEF DESCRIPTION OF THE SEVERAL VIEWS OF THE DRAWINGS

These drawings are exemplary illustrations of certain embodiments and, as such, are not intended to limit the disclosure.

FIG. 1 illustrates a prior art exemplary atherectomy device.

FIG. 2 illustrates a prior art exemplary plot of a stall condition with subsequent management of the driving motor.

FIG. 3 illustrates a prior art exemplary plot of a stall condition with subsequent management of the driving motor.

FIG. 4A illustrates an exemplary drive shaft damaged after a stall condition.

FIG. 4B illustrates an exemplary drive shaft damaged after a stall condition.

FIG. 5 is a schematic of one embodiment of the present invention.

FIG. 6 is an exemplary embodiment of a dynamic braking.

FIG. 7 is an exemplary embodiment of a plot illustrating motor current and rotational speed with an upper threshold for current and rate of change of current.

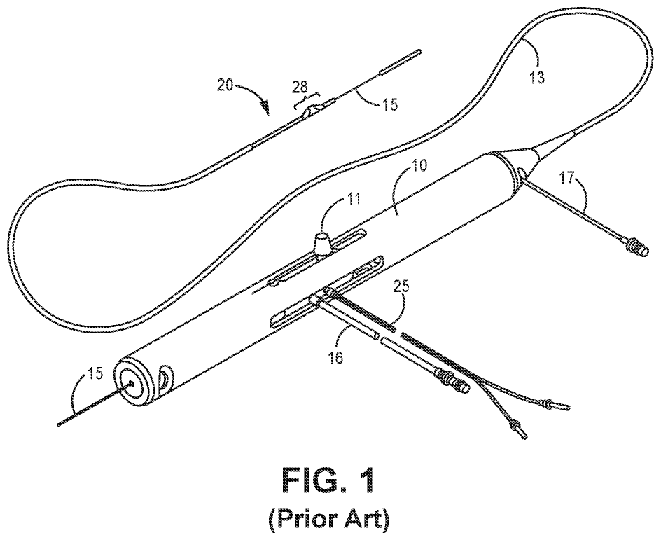

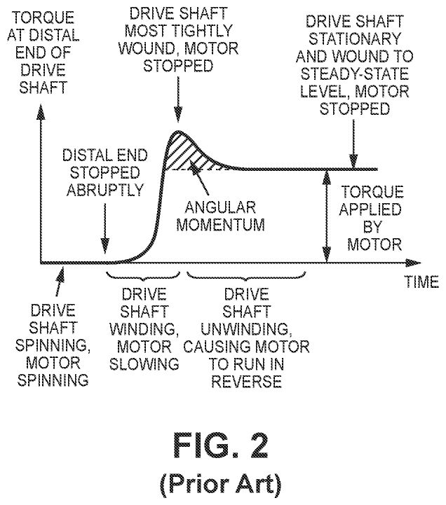

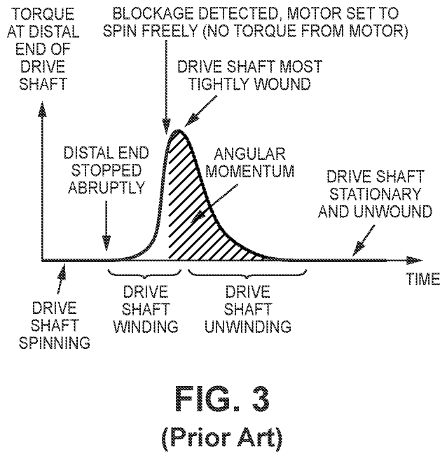

### DETAILED DESCRIPTION OF THE INVENTION

With reference to the Figures, various control mechanisms are disclosed after a stall condition is detected in order to safely and predictably release the stored rotational energy and angular momentum within a stalled, and wound, drive shaft.

In all embodiments, the control mechanism detects a stall condition using one or more sensors configured to sense an operational parameter. Such a stall condition may be determined by using an operational parameters sensed by one or more of a current sensor, a voltage sensor and/or an applied torque sensor wherein a predetermined upper threshold limit for a sensed operational parameter is fixed within the system's controller. If that upper threshold limit for one or more of motor current, rate of change of motor current, motor voltage, rate of change of motor voltage, torque applied by the motor to the drive shaft, and rate of change of applied torque is exceeded, a stall condition may be declared as detected.

In addition, a rotational speed and/or rotational position sensor may be used, alone or in combination with one or more of the current sensor, voltage sensor and/or applied torque sensor, and a predetermined lower limit which in a stall condition is zero rpms. An exemplary rotational speed and/or rotational position sensor comprises a hall sensor. The rotational speed operational parameter may be sensed at the electric motor, the drive shaft and/or the distally positioned atherectomy tool. If the sensed rotational speed drops to zero rpms, then a stall condition is declared as detected.

Some exemplary stall detection methods follow:

1. The sensed electric motor current exceeds a fixed upper current threshold instantly, i.e., once, or for a period of time.

The predetermined, fixed threshold varies with each atherectomy device model. For example, more current for larger atherectomy tools, or for more than one atherectomy tool disposed along a drive shaft, and/or longer drive shafts during normal operation. These variables may be readily predetermined and characterized for a customized threshold applicable to each atherectomy device model and may be stored within the atherectomy controller at the processor and/or memory, or may be entered at a keyboard operatively connected to the controller.

2. Motor current rate of change di/dt exceeds a fixed threshold instantly, i.e., once, or for a period of time.

3. Combination of sensed operational parameters motor speed and motor current.

Detect a stall condition if motor speed drops by some amount (fixed or percentage or rate of change dv/dt) and motor current rises by some amount (fixed or percentage or rate of change di/dt) at the same time for a predetermined period of time.

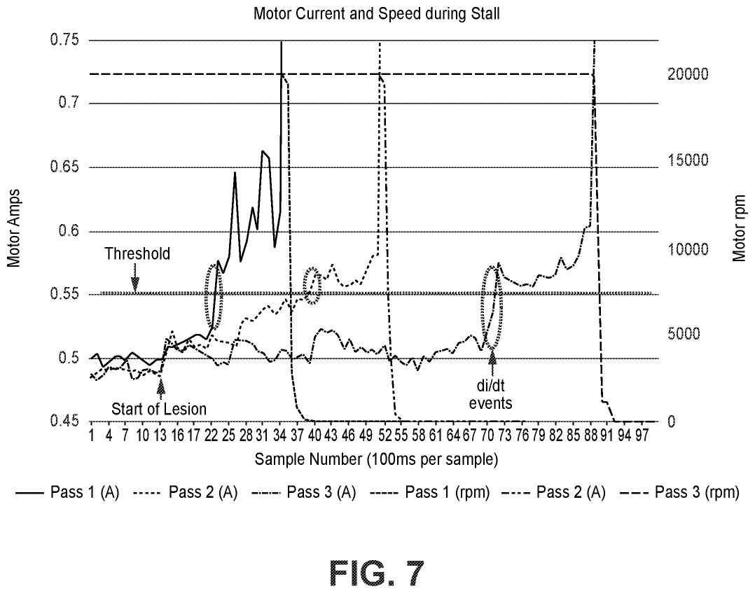

FIG. 7 illustrates the above concepts in exemplary embodiments with experimental rotational speed and current data. The horizontal dashed line of FIG. 7 represents an exemplary fixed upper current threshold as described above in (1), while the dashed ovals show over-threshold limit rate of current change (di/dt) events as described above in (2) for three separate passes of the exemplary atherectomy tool through a lesion.

One advantage of the current threshold or di/dt approach is each of them may detect the stall at an earlier point in time as compared with a rotational speed threshold limit approach.

### Embodiment 1—Stall Detected, Dynamic Electric Motor Braking to Control Drive Shaft Unwind

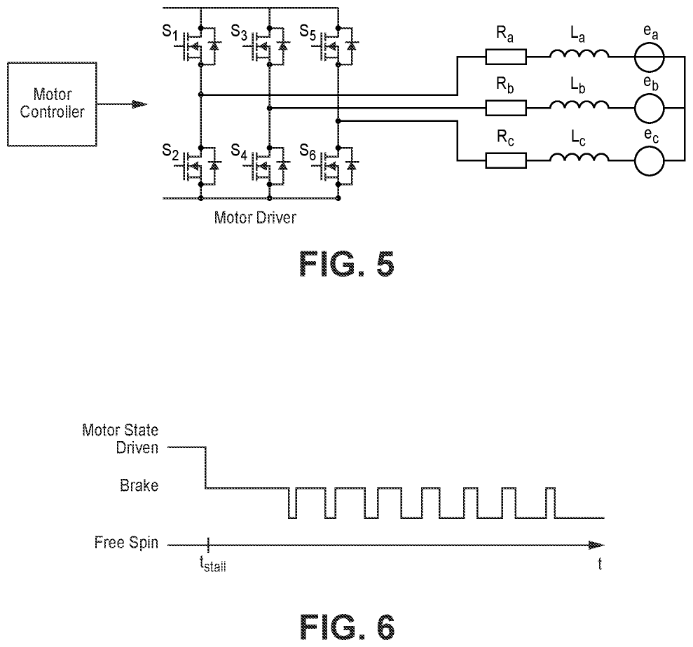

In this embodiment, the motor driver outputs are configured to tie the motor windings (typically there are 3 windings). The basic schematic of the system is illustrated in FIG. 5 where three winding structures are provided within the BLDC motor, each winding structure comprising electrical components labeled R (resistor), L (inductor) and e (back emf). After a stall condition is detected as discussed above, the motor controller signals all of the low-side switches (S₂, S₄, S₆) in the motor driver to turn on. This switching shorts all three of the motor windings together, causing the back emf of the motor to resist rotation, placing the motor into a braking condition. This braking condition configuration results in a situation wherein the faster the motor tries to turn, the higher the magnitude of the back emf and, in turn, the more braking resistance is provided. Thus, the back emf of the motor is directly related to the speed at which the motor tries to rotate. And, the braking force is directly related to the back emf of the motor and to the speed at which the motor tries to rotate when configured in the braking condition.

As shown in FIG. 6, after detecting a stall condition at tₛₜₐₗₗ the braking condition may be initiated. In some embodiments, the braking condition may be instructed by the controller for a predetermined amount of time, then the braking condition may be instructed by the controller to be released for a predetermined amount of time. As shown, there are a plurality of braking condition periods with intervening non-braking condition periods (wherein the motor commences rotational turning in the reverse direction). In FIG. 6, the braking condition periods become progressively longer, and the non-braking or motor driven condition periods become progressively shorter, as time goes on. At the initiation of the dynamic braking of FIG. 6, the first braking condition period is longer than the first non-braking (motor reversing) condition period. This situation eventually reverses itself so that near the end of the dynamic braking, the braking condition periods become longer than the non-braking (motor reversing) condition periods. In other embodiments, the braking and non-braking conditions may be of equivalent lengths over the length of time dynamic braking is in effect. Alternatively, other embodiments of dynamic braking may comprise the initial braking periods to be longer than the initial non-braking periods, with braking periods increasing in time and non-braking periods shortening over time as the dynamic braking proceeds.

The skilled artisan will recognize that any combination of periods for the braking and non-braking conditions may be employed. In addition, the number of braking and non-braking conditions within a dynamic braking may be 50 or less, 40 or less, 30 or less, 20 or less, or 10 or less.

In some embodiments, a sensor may be employed to sense and determine if the unwinding of the drive shaft is complete. This may be a torque sensor and/or rotational speed sensor or the like. In this embodiment, after completing dynamic braking comprising predetermined braking and non-braking periods and numbers of braking and non-braking conditions as described above, an intervening free-spinning period may be initiated by the controller whereby the drive shaft is released from the motor's applied torque for a very short period of time. During this free-spinning period, the sensor monitors the drive shaft for any further reverse rotational movement. If no reverse rotational movement is detected or sensed, then the controller determines that the unwinding is complete and no further dynamic braking is required. If reverse rotational movement is detected or sensed, then the controller may apply a supplemental dynamic braking that may comprise the same braking and non-braking periods and numbers as the initially applied dynamic braking. In other embodiments, the supplemental dynamic braking may differ in the braking and non-braking periods and numbers. For example, a single braking condition followed by a single non-braking condition may be applied, followed by another free-spinning evaluation. This process may be repeated until the controller determines that the drive shaft is fully unwound.

### Embodiment 2—Stall Detected, Motor Slowly Reversed a Predetermined Number of Rotations

In this embodiment, when a stall is detected as described above, the controller signals or instructs the motor driver to stop applying forward torque to the drive shaft and to apply a reverse torque to the drive shaft for a predetermined number of turns or rotations. The torque applied to reverse the drive shaft is less than the applied torque in the forward direction. The predetermined number of reversing turns or rotations may be 50 or less, 40 or less, 30 or less, 20 or less or 10 or less. In certain embodiments, the drive shaft winding and unwinding may be characterized and a predetermined number of reversing turns may be determined for one or more types or models of drive shafts. These data may be stored within a memory that is electrically or operably connected with the motor controller for execution.

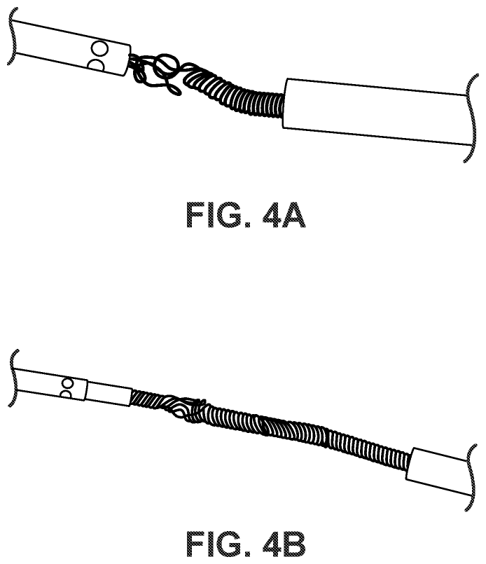

As seen in FIGS. 4A and 4B, the wire filars of the drive shaft may be damaged in a stall condition. Individual drive shaft models may be characterized to determine appropriate operating limits to aid in ensuring that drive shaft damage during stall conditions does not occur. For example, drive shaft damage can occur if the motor current limit is set too high, thus allowing torque to be applied to the drive shaft at a level that is unsafe. The characterization data may be used to help establish predetermined limits of the sensed parameters of the electric motor relating to, e.g., current, voltage, applied torque and/or rotational speed, wherein the predetermined limits used during an atherectomy procedure are customized for a particular model of drive shaft. In addition, the dynamic braking parameters may be customized and predetermined for individual models of drive shafts. These customized and predetermined data may be stored within the controller, e.g., in the memory or processor, for instructed execution at the appropriate time, e.g., initial normal operation and/or following stall detection.

The basic characterization method steps for a drive shaft characterization follow:
1. Select a relatively low current limit, one that is below a known upper threshold limit for current.
2. Initiate rotation of the drive shaft with an electric motor, and induce stalling of the distal end of the drive shaft, wherein the distal end is prevented from rotating.
3. Measure the number of motor rotations that occur as the drive shaft winds up, and before the electric motor stalls.
4. Measure the number of motor rotations as the drive shaft unwinds, releasing the stored rotational energy and angular momentum, after the motor stalls.
5. Calculate the difference between the measured wind-up rotations and the measured unwind rotations.
6. Increase the motor current incrementally and repeat steps 2-5.
7. Compare the difference from step 5 against a predetermined upper threshold that is established using visual damage as an indicator, wherein the predetermined upper threshold is established to be less than the visual damage current level.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. A rotational atherectomy system, comprising:
an elongated flexible drive shaft comprising coiled wires;
an atherectomy tool disposed at or near a distal end of the drive shaft;
an electric motor rotationally connected with a proximal end of the drive shaft, wherein the electric motor is configured to apply torque to, and rotate, the drive shaft and the atherectomy tool in a first rotational direction;
a controller operatively connected with the electric motor, the controller comprising a memory, a processor comprising preprogrammed executable instructions and in operative connection with the memory;
at least one sensor operatively coupled with the motor, each one of the at least one sensor providing a sensed signal corresponding with an operational parameter of the electric motor, the sensor in operative communication with the controller,
wherein the controller is configured to use each one of the sensed signals to detect when a stall condition has occurred,
wherein when a stall condition is detected, the controller is configured to stop the electric motor from applying torque to the drive shaft and to cause the electric motor to execute a dynamic braking comprising a one or more braking conditions and one or more non-braking conditions comprising applying torque to the drive shaft in a second rotational direction opposite that of the first rotational direction,
wherein each braking condition is followed by a non-braking condition.

2. The rotational atherectomy system of claim 1, wherein the at least one sensor is selected from one or more of the group consisting of: current sensor, voltage sensor, applied torque sensor, and rotational speed sensor.

3. The rotational atherectomy system of claim 2, wherein the sensed signal corresponding with an operational parameter of the electric motor is selected from one or more of the group consisting of: current, rate of change of current, voltage, rate of change of voltage, applied torque, rate of change of applied torque, rotational speed, and rate of change of rotational speed.

4. The rotational atherectomy system of claim 1, wherein a first braking condition comprises a time period that is less than the time period of any subsequent braking condition.

5. The rotational atherectomy system of claim 4, wherein the time period of the braking conditions increases successively.

6. The rotational atherectomy system of claim 1, wherein a first non-braking condition comprises a time period that is longer than the time period of subsequent non-braking conditions.

7. The rotational atherectomy system of claim 6, wherein the time period of each non-braking condition increases successively.

8. The rotational atherectomy system of claim 1, wherein the motor comprises a plurality of motor windings in switched communication with low-side switches, wherein when a stall is detected, the controller is configured to actuate all of the low-side switches and to cause a back emf of the motor to resist rotation.

9. The rotational atherectomy system of claim 8, wherein actuation of the low-side switches results in a braking condition.

10. The rotational atherectomy system of claim 3, wherein following execution of at least one braking condition and non-braking condition, the controller is configured to instruct the electric motor to remove all torque applied to the drive shaft and to allow the drive shaft to freely rotate.

11. The rotational atherectomy system of claim 10, wherein the at least one sensor monitors whether the drive shaft rotates in the second rotational direction and wherein the controller is configured to determine if the drive shaft is fully unwound or not fully unwound.

12. The rotational atherectomy system of claim 11, wherein if the drive shaft is determined to not be fully unwound, the controller is configured to instruct the electric motor to execute at least one dynamic braking cycle comprising a braking condition followed by a non-braking condition.

13. The rotational atherectomy system of claim 1, wherein the dynamic braking comprises a predetermined number of braking conditions and non-braking conditions.

14. The rotational atherectomy system of claim 5, wherein the dynamic braking comprises predetermined time periods for each braking condition.

15. The rotational atherectomy system of claim 7, wherein the dynamic braking comprises predetermined time periods for each non-braking condition.

16. The rotational atherectomy system of claim 13, wherein the dynamic braking is customized and predetermined for an individual model of drive shaft.

17. The rotational atherectomy system of claim 16, wherein the customized and predetermined dynamic braking for one or more individual models of drive shafts is stored within the memory or processor for instructed execution by the electric motor.

18. The rotational atherectomy system of claim 3, further comprising predetermined threshold limits for one or more of the group consisting of: motor current, rate of change of motor current, motor voltage, rate of change of motor voltage, applied torque by the motor to the drive shaft rate of change of applied torque by the motor to the drive shaft, motor rotational speed, and rate of change of motor rotational speed, wherein a stall condition is detected if one or more of the predetermined threshold limits is exceeded.

19. The rotational atherectomy system of claim 3, further comprising a predetermined threshold limit for motor current, wherein a stall condition is detected if the predetermined threshold limit for motor current is exceeded.

20. The rotational atherectomy system of claim 3, further comprising a predetermined threshold limit for motor current rate of change, wherein a stall condition is detected if the predetermined threshold limit for motor current rate of change is exceeded.

21. The rotational atherectomy system of claim 3, further comprising a predetermined threshold limit for motor current, and a predetermined threshold limit for motor current rate of change, wherein a stall condition is detected if the predetermined threshold limit for motor current and the predetermined threshold limit for motor current rate of change are both exceeded.

22. The rotational atherectomy system of claim 3, further comprising a predetermined threshold limit for motor current, and a predetermined threshold limit for motor rotational speed, wherein a stall condition is detected if the predetermined threshold limit for motor current and the predetermined threshold limit for motor rotational speed are both exceeded.

23. The rotational atherectomy system of claim 3, further comprising a predetermined threshold limit for motor current rate of change, and a predetermined threshold limit for motor rotational speed, wherein a stall condition is detected if the predetermined threshold limit for motor current rate of change and the predetermined threshold limit for motor rotational speed are both exceeded.
